SEQUENCE LISTING (4 sequences)

SEQ ID NO: 1 - protein, 11 aa, Synthetic construct
DGRARQSQDDD

SEQ ID NO: 2 - protein, 11 aa, Synthetic construct
GSQIALRRRDF

SEQ ID NO: 3 - protein, 5 aa, synthetic construct
TYGIQ

SEQ ID NO: 4 - protein, 17 aa, synthetic construct
WIHAGTGGTKYSRKFQG

(19) United States
(12) Patent Application Publication
(10) Pub. No.: US 2025/0368755 A1
(43) Pub. Date: Dec. 4, 2025

(54) PEPTIDE THAT SPECIFICALLY BINDS TO PPC REGION OF SARS-COV-2 SPIKE PROTEIN AND COMPOSITION FOR PREVENTING SARS-COV-2 INFECTION USING THE SAME

(71) Applicant: UIF (University Industry Foundation), Yonsei University, Seoul (KR)

(72) Inventor: Jae-Chul PYUN, Seoul (KR)

(73) Assignee: UIF (University Industry Foundation), Yonsei University, Seoul (KR)

(21) Appl. No.: 19/218,958

(22) Filed: May 27, 2025

(30) Foreign Application Priority Data
May 29, 2024 (KR) 10-2024-0069899

(51) Int. Cl. C07K 16/40; A61P 31/14; C07K 7/06

(52) U.S. Cl. CPC C07K 16/40; A61P 31/14; C07K 7/06; C07K 2317/565; C07K 2317/76; C07K 2317/92

(57) ABSTRACT

Disclosed are a peptide specifically binding to a PPC region of a SARS-CoV-2 spike protein and a composition for preventing SARS-CoV-2 infection using the same. The peptide includes a peptide sequence of DGRARQSQDDD or GSQIALRRRDE.

### CROSS-REFERENCE TO RELATED APPLICATION(S)

This application claims the benefit under 35 USC 119 (a) of Korean Patent Application No. KR 10-2024-0069899 filed on May 29, 2024, with the Korean Intellectual Property Office, the disclosures of which are incorporated herein by reference in their entirety.

### INCORPORATION-BY-REFERENCE OF MATERIAL SUBMITTED BY U.S.P.T.O. EFS-WEB

This application contains a Sequence Listing, which is being submitted in computer readable form via the United States Patent and Trademark Office Patent Center and which is hereby incorporated by reference in its entirety for all purposes. The XML file submitted herewith, which is named as “NewApp_1710950012_SequenceListing” and is created on May 27, 2025, contains a 4.61 KB file.

### BACKGROUND

### Field

The present disclosure relates to a peptide specifically binding to a PPC region of a SARS-CoV-2 spike protein and a composition for preventing SARS-CoV-2 infection using the same.

### Description of Related Art

The infection of SARS-CoV-2 was known to begin by the binding of spike protein (SP) of SARS-CoV-2 to an angiotensin-converting enzyme 2 (ACE2) receptor. For the further infection of SARS-CoV-2 into host cell membrane, the cleavage of SP was required at two regions called S1/S2, and S2′ which were called proprotein convertase (PPC) sites. Further, the proteolytic enzymes called furin and TMPRSS2 are known to cleave S1/S2, and S2′ respectively. Because these regions were known to have highly conserved amino acid sequence among corona virus as shown in FIG. 1A, the antibodies with the binding affinity to these regions have been developed to prevent the infection of SARS-CoV-2. However, the approach to these regions were known to be limited for antibodies because of steric hindrance from the structure of SP around the PPC region. To overcome the steric hindrance to approach the PPC region, small molecules with the binding affinity to these regions have been also developed as pan-corona drugs by many research groups.

### SUMMARY

In the present disclosure, Fv-antibodies with a high binding affinity to the PPC region were screened from Fv-antibody library to prevent the infection of SARS-CoV-2. The Fv antibodies were screened from Fv-antibody library using the amino acid sequence of PPC region including two cleavage sites as a screening probe. The screened Fv-antibodies were expressed as soluble protein and binding activity to PPC region was estimated. Finally, the neutralizing activity of Fv-antibodies were estimated using pseudo-virus particles with spike proteins of four different SARS-CoV-2 variants of Wu-1 (wild type), Delta, Omicron (BA.2), and Omicron (BA.4/5).

A purpose to be achieved by the present disclosure is to effectively block the infection route of SARS-CoV-2 virus and to strengthen the binding and neutralization mechanisms of antibodies in the process prior to the invasion of the virus into cells. The peptide developed for this purpose binds to a specific site of the virus's spike protein, thereby providing the potential to interfere with the entry of the virus into the cell. In particular, this peptide may play a key role in the early invasion phase of the virus by interfering with the interaction between the virus and the receptor on the cell surface.

In one aspect, the present disclosure provides a peptide capable of specifically binding to a PPC region of a SARS-CoV-2 spike protein, wherein the peptide includes a peptide sequence of SEQ ID NO: 1 or a peptide sequence of SEQ ID NO: 2:

[SEQ ID NO: 1]
DGRARQSQDDD
[SEQ ID NO: 2]
GSQIALRRRDF.

In one embodiment, the peptide may bind to the PPC region of the SARS-CoV-2 spike protein, thereby preventing the cleavage of the spike protein by TMPRSS2 of an infection target host cell.

Another aspect of the present disclosure provides a nucleic acid coding the peptide as described above.

Still another aspect of the present disclosure provides a recombinant expression vector including the nucleic acid as described above.

Still yet another aspect of the present disclosure provides a cell transformed with the recombinant expression vector as described above.

In one embodiment, the cell includes at least one cell selected from the group consisting of animal cells, plant cells, yeast, E. coli, and insect cells.

In one embodiment, the cell includes at least one cell selected from the group consisting of COS7 (monkey kidney cells) cells, NSO cells, SP2/0 cells, CHO (Chinese hamster ovary) cells, W138, BHK (baby hamster kidney) cells, MDCK, myeloma cell lines, HuT 78 cells and HEK293 cells, Escherichia coli, Bacillus subtilis, Streptomyces sp., Pseudomonas sp., Proteus mirabilis or Staphylococcus sp., Aspergillus sp., Pichiapastoris, Saccharomyces cerevisiae, Schizosaccharomyces sp., and Neurosporacrasa.

Still yet another aspect of the present disclosure provides an antibody or antigen-binding fragment thereof capable of specifically binding to a PPC region of a SARS-CoV-2 spike protein, wherein the antibody includes a Fv antibody, wherein a CDR3 region of the Fv antibody comprises a peptide sequence of SEQ ID NO: 1 or a peptide sequence of SEQ ID NO: 2.

[SEQ ID NO: 1]
DGRARQSQDDD
[SEQ ID NO: 2]
GSQIALRRRDF.

In one embodiment, the antibody or antigen-binding fragment thereof may bind to the PPC region of the SARS-CoV-2 spike protein, thereby interfering with the cleavage of the spike protein by TMPRSS2 of the infection target host cell.

Still yet another aspect of the present disclosure provides a composition for preventing SARS-CoV-2 infection, the composition comprising the antibody or an antigen-binding fragment thereof as described above.

Specifically, Fv-antibodies against PPC region of SARS-CoV-2 spike protein were screened from Fv-antibody library to prevent SARS-CoV-2 infection. Two screened Fv-antibodies were expressed as GPF labeled proteins and the binding affinity was estimated. The binding regions of two Fv-antibodies were analyzed to be corresponding to the cleavage sites of furin and TMPRSS2. The neutralizing activity of two Fv-antibodies were demonstrated using cell-based infection assay using SARS-CoV-2 pseudo-viruses.

An effect of the present disclosure is that the peptide binds to the specific site of the SARS-CoV-2 spike protein to interfere with the interaction between the virus and the cell surface receptor, thereby providing the possibility of effectively blocking the influx of the virus into the cells. This mechanism may provide a wide range of protective effects in response to variants even in the current situation where the variation of the virus is still in progress.

Effects of the present disclosure are not limited to the effects mentioned above, and other effects not mentioned will be clearly understood by those skilled in the art from the description as set forth below.

In addition to the above effects, specific effects of the present disclosure are described together while describing specific details for carrying out the present disclosure.

### BRIEF DESCRIPTION OF DRAWINGS

FIG. 1A is a diagram comparing spike protein cleavage positions and amino acid sequences of various coronavirus and variants thereof with each other.

FIG. 1B is a diagram showing the spike protein structure of SARS-CoV-2 and cleavage positions by Furin and TMPRSS2.

FIG. 1C is a flow diagram illustrating a process of aligning and validating candidate clones selected using a PPC probe in a Fv-antibody library.

FIG. 1D is a diagram showing a result of measuring fluorescence intensities of the selected clones and comparing and analyzing the effects thereof.

FIG. 2A is a diagram showing the structure of an Fv-antibody expression plasmid describing the cloning, expression, and purification process of Fv-antibody.

FIG. 2B is a diagram that visualizes the binding of Fv-antibody to the SARS-CoV-2 PPC region and provides analytical data for measuring binding efficiency.

FIG. 2C is a diagram showing structural data analyzing amino acids in the PPC region of SARS-CoV-2 with which two types of Fv-antibodies (Fv1 and Fv2) interact, and binding energies thereof.

FIG. 2D is a diagram showing experimental results showing how Fv-antibodies interfere with cleavage of the PPC region through Furin and TMPRSS2 cleavage experiments.

FIG. 3A is a diagram showing an experimental process of visualizing an infection suppression effect when an Anti-PPC Fv antibody binds to SARS-CoV-2 pseudo-virus particles.

FIG. 3B is a diagram comparing and analyzing the anti-infection effects of Anti-PPC Fv antibodies on several variations of SARS-CoV-2 (wild-type, delta, omicron BA.2 and BA.4/5) based on fluorescence images.

FIG. 3C is a diagram showing a result of statistically comparing the infection suppression efficiency of Anti-PPC Fv1 and Fv2 antibodies on each of several variations of SARS-CoV-2.

FIG. 4 is a diagram showing the binding process of the Fv antibody to a specific site of SARS-CoV-2 spike protein.

### DETAILED DESCRIPTIONS

Advantages and features of the present disclosure, and a method of achieving the advantages and features will become apparent with reference to embodiments described later in detail together with the accompanying drawings. However, the present disclosure is not limited to the embodiments as disclosed under, but may be implemented in various different forms. Thus, these embodiments are set forth only to make the present disclosure complete, and to completely inform the scope of the present disclosure to those of ordinary skill in the technical field to which the present disclosure belongs, and the present disclosure is only defined by the scope of the claims.

Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure. Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, and “including” when used in the present disclosure, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of associated listed items. Expression such as “at least one of” when preceding a list of elements may modify the entire list of elements and may not modify the individual elements of the list. In interpretation of numerical values, an error or tolerance therein may occur even when there is no explicit description thereof.

In interpreting a numerical value, the value is interpreted as including an error range unless there is no separate explicit description thereof. In the context of the present disclosure, the term “about” may mean about ±1%, about ±2%, about ±3%, about ±4%, about ±5%, about ±6%, about ±7%, about ±8%, about ±9%, or about ±10% of a value stated herein.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

A peptide according to an embodiment of the present disclosure is a peptide capable of specifically binding to the PPC region of the SARS-CoV-2 spike protein, and may include the peptide sequence of SEQ ID NO: 1 or the peptide sequence of SEQ ID NO: 2:

[SEQ ID NO: 1]
DGRARQSQDDD
[SEQ ID NO: 2]
GSQIALRRRDF.

In the context of the present disclosure, the dictionary meaning of SARS-CoV-2 is a virus that causes a novel coronavirus infection, and is a type of coronavirus that causes acute respiratory syndrome. In the context of the present disclosure, the dictionary meaning of the spike protein of SARS-CoV-2 is a surface protein that plays an important role when the virus binds to and invades into the host cell. This protein protrudes from the surface of the virus particle and mediates the binding of the virus to the specific receptor such as ACE2 in the host cell. In the context of the present disclosure,

In the context of the present disclosure, the meaning of the ACE2 receptor is an acronym of an angiotensin-converting enzyme 2, which is a protein present on a host cell surface. The spike protein of SARS-CoV-2 virus binds to this receptor. Thus, this receptor plays an important role in the process of the virus invasion into cells. That is, this receptor enables the infection by providing a pathway for entry of the virus into the cells. The ACE2 is widely distributed on the cell surface of several organs, mainly the heart, kidneys, and lungs. Via the binding of the SARS-CoV-2 virus to this receptor, the SARS-CoV-2 enters the host cell and begins replication therein. Thus, directly inhibiting the binding of the spike protein of SARS-CoV-2 to this receptor, or by indirectly inhibiting the binding by blocking the required mechanism prior to the binding may allow the host cell to be prevented from being infected with the SARS-CoV-2.

In the context of the present disclosure, the dictionary meaning of the PPC region of the spike protein of SARS-CoV-2 means the proprotein convertase site, where cleavage of the spike protein by proteases required to promote viral activation and entry into host cells occurs. In an embodiment, the peptide may bind to the PPC region of the SARS-CoV-2 spike protein, thereby interfering with the cleavage of the spike protein by TMPRSS2 of the infection target host cell. In the context of the present disclosure, the meaning of TMPRSS2 is defined as one of the cell surface proteases that aid in the penetration of the virus into the host cell. This enzyme plays an essential role in cleaving the spike protein of the SARS-CoV-2 virus at a specific location, thereby activating the virus to fuse with the host cell membrane such that the virus eventually enters the cell.

In one example, a nucleic acid according to an embodiment of the present disclosure may encode the peptide. In the context of the present disclosure, the dictionary meaning of a nucleic acid is a biochemical substance that stores and conveys genetic information, and includes DNA or RNA. These nucleic acids are components of genes and contain information necessary for the biosynthesis of proteins and peptides. In the context of the present disclosure, the dictionary meaning of the nucleic acid encoding the peptide means that the nucleic acid (DNA or RNA) has genetic information that instructs the synthesis of a peptide composed of a specific amino acid sequence. This information is converted into the peptide via transcription and translation processes. During the transcription process, the genetic information of DNA is copied into mRNA, and during the translation process, the code of mRNA is translated into an amino acid sequence by the ribosome, resulting in the synthesis of the peptide. Through this process, the nucleic acid directly plays a role in determining the structure and function of the peptide.

As known in the art, a combination of nucleic acids encoding the amino acid included in the peptide may vary. Accordingly, according to the present disclosure, there are proposed not only the above-described peptide but also a nucleic acid encoding the above-described peptide, wherein the nucleic acid includes all of theoretical 221,184 nucleic acid sequences encoding the SEQ ID NO: 1 and theoretical 2,985,984 nucleic acid sequences encoding the SEQ ID NO: 2. These diverse nucleic acid sequences are attributed to the codon variability of the amino acids. A codon is three consecutive bases of the nucleic acid encoding one amino acid, and multiple codons may encode the same amino acid, so that various nucleic acid sequences for encoding the same peptide sequence may be present. This increases the flexibility of the subject matter of the present disclosure and may help to select nucleic acid sequences optimized for specific biological systems or applications. For example, the nucleic acid sequences may be optimized by taking into account use of the codon that is translated more efficiently in certain species of organisms. This may contribute to enhancing the expression level and stability of the peptide.

In one example, a recombinant expression vector according to an embodiment of the present disclosure may include the nucleic acid. In the context of the present disclosure, the dictionary meaning of the recombinant expression vector is a molecule used to introduce a gene into another cell to express a protein or a peptide. This vector may take the form of plasmids, viruses, artificial chromosomes, etc., and includes essential elements for replication and gene expression, such as selection markers, promoters, reporter genes, etc.

Since the recombinant expression vector includes the nucleic acid, the efficiency and specificity of gene expression may be greatly improved. The nucleic acid incorporated within this vector directs the production of a target protein or peptide within a specific cell type, and thus may be used in medical treatment as well as in scientific research. For example, it plays an important role in a variety of applications, including disease modeling, gene therapy, vaccine development, and mass production of biological agents. In particular, according to the present disclosure, since the specific peptide exhibits high binding specificity to the target protein, the vector corresponding thereto may be an effective tool for preventing or treating SARS-CoV-2 virus infection by optimizing the expression of the peptide.

In one example, a cell according to an embodiment of the present disclosure may be transformed with the recombinant expression vector. In the context of the present disclosure, the dictionary meaning of the transformation of the cell is a process of changing the genetic composition of the cell by introducing the DNA from the outside thereto. Through this process, the cell acquires a new gene, which may be expressed in the cell to produce a specific protein.

As the cell is transformed with the recombinant expression vector, continuous production of a specific peptide or protein may be possible. This process allows the cell to receive genetic information and perform a new biological function based on the genetic information. Thus, a purpose of the present disclosure is to produce a peptide that binds to the PPC region of the spike protein of the SARS-CoV-2 virus. The transformed cell may play an important role in a variety of applications, including laboratory studies alone, as well as the production of biologics and the development of gene therapeutics for disease treatment.

In an embodiment, the cell may include one or more cells selected from the group consisting of animal cells, plant cells, yeast, E. coli, and insect cells. In one embodiment, the cells may include one or more cells selected from the group including COS7 (monkey kidney cells) cells, NSO cells, SP2/0 cells, CHO (Chinese hamster ovary) cells, W138, BHK (baby hamster kidney) cells, MDCK, myeloma cell lines, HuT 78 cells and HEK293 cells, Escherichia coli, Bacillus subtilis, Streptomyces sp., Pseudomonas sp., Proteus mirabilis or Staphylococcus sp., Aspergillus sp., Pichiapastoris, Saccharomyces cerevisiae, Schizosaccharomyces sp., and Neurosporacrasa.

Using the various cells as described above, the subject matter of the present disclosure has the flexibility to be applied to a wide range of biological systems. Each cell type has its own physiological characteristics and genetic expression profile, which may help optimize the production, stability, and functionality of peptides. The utilization of these different cell types provides an opportunity to develop optimized expression systems for specific uses. In another aspect, an antibody or antigen-binding fragment thereof according to an embodiment of the present disclosure is an antibody or antigen-binding fragment thereof capable of specifically binding to the PPC region of the SARS-CoV-2 spike protein, wherein the antibody includes the Fv antibody, wherein the CDR3 region of the Fv antibody may include the peptide sequence of SEQ ID NO: 1 or the peptide sequence of SEQ ID NO: 2:

[SEQ ID NO: 1]
DGRARQSQDDD
[SEQ ID NO: 2]
GSQIALRRRDF.

In the context of the present disclosure, the dictionary meaning of an antibody or antigen-binding fragment thereof is a portion of a protein that may bind to, recognize, and neutralize a specific antigen. The antigen-binding fragment of the antibody is generally responsible for binding with the antigen at a specific site within the variable region of the antibody, and the efficacy and specificity thereof are influenced by the amino acid sequence of the binding region.

In one embodiment, the antibody may be a Fv antibody. In the context of the present disclosure, the dictionary meaning of the Fv antibody is a protein comprising two fragments constituting the variable region of the antibody, namely, the variable region (VL) of the light chain and the variable region (VH) of the heavy chain. The Fv antibody has high specificity and binding power, and may be advantageously used as a therapeutic agent or as a diagnostic tool because of its small size.

In an embodiment, the peptide sequence according to SEQ ID NO: 1 or 2 may be included in the CDR3 (Complementarity-Determining Region 3) region of the Fv antibody. The CDR3 region of the Fv antibody is the most variable region of the antibody and plays a decisive role in binding to the antigen. This region is an important factor that allows the antibody to have high affinity and specificity to the specific antigen, and is an important determinant that enables effective binding and recognition of the antibody. Thus, the selection and optimization of the peptide sequences contained in the CDR3 region play an important role in maximizing the function of the antibody.

In an embodiment, the antibody or antigen-binding fragment thereof may bind to the PPC region of the SARS-CoV-2 spike protein, thereby interfering with the cleavage of the spike protein by TMPRSS2 of the infection target host cell. In this way, the antibodies or antigen-binding fragment may offer the potential to effectively inhibit the entry of viruses into the cells. This is to utilize a mechanism of blocking the cleavage process of the spike protein which is essential in the penetration process of SARS-CoV-2 into the cells to prevent the host cell from being infected with the virus. This approach may provide a wide range of protection, especially for mutated viruses, which may be an important basis for the development of antibody therapeutics in response to changes in the virus. Such research may contribute to the search for new strategies in the prevention and treatment of infectious diseases.

In still another aspect, the composition for preventing SARS-CoV-2 infection according to an embodiment of the present disclosure may include the antibody or an antigen-binding fragment thereof as described above. Since the composition for preventing SARS-CoV-2 infection comprises the antibody or antigen-binding fragment thereof, the principle of preventing SARS-CoV-2 infection is that a specific antibody or antigen-binding fragment binds to the spike protein of the virus, in particular the PPC region, thereby interfering with cleavage of this protein by TMPRSS2. This interfering process effectively prevents the virus from fusing with the cell membrane and finally entering the interior of the host cell. Consequently, this composition offers the potential to block the pathway of virus entry into the host cell, thereby preventing infection of SARS-CoV-2 at an early stage.

The composition for preventing SARS-CoV-2 according to an embodiment of the present disclosure includes the antibody or antigen-binding fragment thereof. However, the present disclosure does not exclude the addition of other compositions that may be included in the composition for prevention to the composition for preventing SARS-CoV-2 according to an embodiment of the present disclosure. For example, the composition for prevention may include an adjuvant for enhancing an immune response, a stabilizer and a preservative for maintaining the stability of antibodies and peptides, a solubilizer and a buffer for controlling the pH of the composition and improving solubility thereof, and various immune response enhancing agents for enhancing an immune response. As the adjuvants, substances such as aluminum salts and squalene may be used to increase the immunogenicity against the antigen and provide long-term immune protection. Sugar such as sucrose or trehalose may be used as the stabilizer, and thimerosal (sodium ethylmercurithiosalicylate) or paraben may be used as the preservative. In addition, buffer solutions such as phosphate buffer solution (PBS) or tris buffer are used to adjust the pH of the composition and improve solubility thereof. The immune response enhancing agent may contribute to enhancing immune memory and increasing protective effects.

Hereinafter, examples of the present disclosure will be described. However, the examples as described below are only some implementations of the present disclosure, and the scope of the present disclosure is not limited to the following examples.

### Materials and Methods

### Materials

SARS-CoV-2 PPC region (residues: 661-900, 53.6 kDa), and TMPRSS2 region (residues: 106-492, 69.7 kDa) were custom-synthesized with the GFP by Cosmo Genetech (Seoul, South Korea). Luria-Bertani medium was obtained from Duchefa (Haarlem, Netherlands). DNA oligonucleotides for Fv-antibody library production were synthesized by Bionics (Seoul, South Korea). Furin was obtained from Sigma Aldrich Inc. (St. Louis, MO, USA). The materials related to SARS-CoV-2 variants pseudo-virus production have been described in previous study. Pseudotyping SP variant vectors and HEK Blue™ cells (overexpressing hACE2-TMPRSS2) were obtained from InvivoGen Inc. (San Diego, CA, USA). Dulbecco's modified Eagle's medium and Opti-MEM were obtained from Gibco Inc. (Waltham, MA, USA).

### Screening of Anti-PPC Fv From Fv-Antibody Library

Preparation of the Fv-antibody library has been described previous studies. Clones exhibiting binding activity to the PPC region probe were screened from the Fv-antibody library as follows: (1) The randomized CDR3 region of Fv-antibody was expressed on the membrane of E. coli. (2) PPC region probe (1 μM, 100 μL) was treated with the Fv-antibody library (100 μL) for 1 h at 37° C. (3) After washing with PBS containing 0.01% Tween 20, screened clones with binding activity were sorted (n=500) using a flow cytometer (FACSCalibur™, NJ, USA). (4) The screened clones were selected after CDR3 region identification by DNA oligonucleotide sequencing.

### The KD Estimation of Anti-PPC Fv

The KD of the Anti-PPC Fv was determined using a SPR biosensor (i-Cluebio, Seongnam, South Korea). The SPR chips, composed of BK-7 glass, were coated with a 2 nm titanium layer followed by a 48 nm gold layer. Immobilization of Anti-PPC Fv onto the gold surface was performed. Anti-PPC Fv (20 μg/mL, 100 μL) was incubated on the SPR chips for 16 h at 4° C. Subsequently, the SPR chips were blocked using BSA (1 mg/mL) for 1 h at 37° C. After washing with PBS containing 0.01% Tween 20, PPC antigen concentration in the range of 12.5 to 100.0 nM was introduced at a flow rate of 25 μL/min for 10 min to promote binding. Thereafter, in order to facilitate dissociation, the washing buffer (PBS) was flowed for 10 min at the same rate.

### Furin Cleavage Inhibition Assay

To analyze whether the anti-PPC Fv binds to the PPC region and inhibits furin cleavage, the Fv-antibody was incubated with a fixed concentration of PPC and furin. After mixing PPC (100 μg/mL) with furin (20 units), the Anti-PPC Fv was added at concentrations ranging from 1 to 2,500 nM and incubated at 37°° C. for 1 h. Subsequently, the reacted samples were analyzed using SDS-PAGE gel, and the band intensity was quantified using ImageJ software.

### Production of Pseudo-Viral Particles

Production of SARS-CoV-2 SP variants pseudo-virus (Wu-1, Delta, Omicron BA.2, and Omicron BA.4/5) was carried out using Lenti-X™ HEK293T cells. The generation of pseudo-viral particles followed these steps:
(1) Lenti-X™ HEK cells (1×105 cells) were cultured in 15 mL of DMEM containing with 10% FBS for 1 days.
(2) Transfection reagent (FuGENE, 10 μL) was mixed with 5 μg of each plasmid (SARS-CoV-2 SP pseudotyping vectors, pLVXS-ZsGreen1-Puro, and psPAX2) in 1 mL of Opti-MEM and incubated at 37° C. for 10 min.
(3) The medium was replaced with 10 mL of DMEM containing 10% FBS, and the mixture (1 mL of Opti-MEM) from step 2 was added to the cells.
(4) After 3 days of incubation, the supernatant was collected by centrifugation (500×g, 10 min).
(5) Lenti-X™ concentrator was added to the supernatant and incubated for 3 h at 4° C.
(6) The particles were collected by centrifugation (1,500×g, 45 min) and supplemented with DMEM.
(7) The titers of the pseudo-viral particles were estimated using Lenti-X™ qRT-PCR, yielding results between 108 and 109 copies/mL.

### Neutralization Assay Using Anti-PPC Fv

The neutralizing activity of the screened Anti-PPC Fv was evaluated through an in vitro cell-based infection assay. In this assay, HEK-Blue™ cells (overexpressing hACE2-TMPRSS2, 5.0×10⁴/well) were plated onto 96-well microplates coated with poly L-lysine and incubated for 1 day. The Anti-PPC Fv (20 ↑g/mL) was then incubated with the pseudo-viral particles for 30 min at 37° C. in 100 μL of DMEM containing 2% FBS. Subsequently, the pseudo-viral particles preincubated with Anti-PPC Fv were added to HEK-Blue™ cells and incubated for 48 h. Cell images were captured using a fluorescence microscope (Eclipse Ts2).

### Screening of Fv-Antibodies Against PPC Site

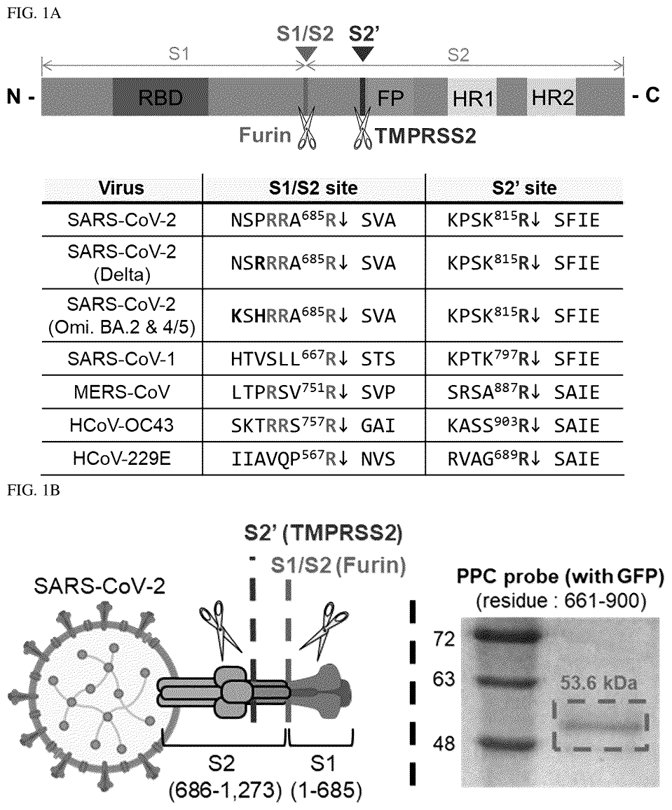

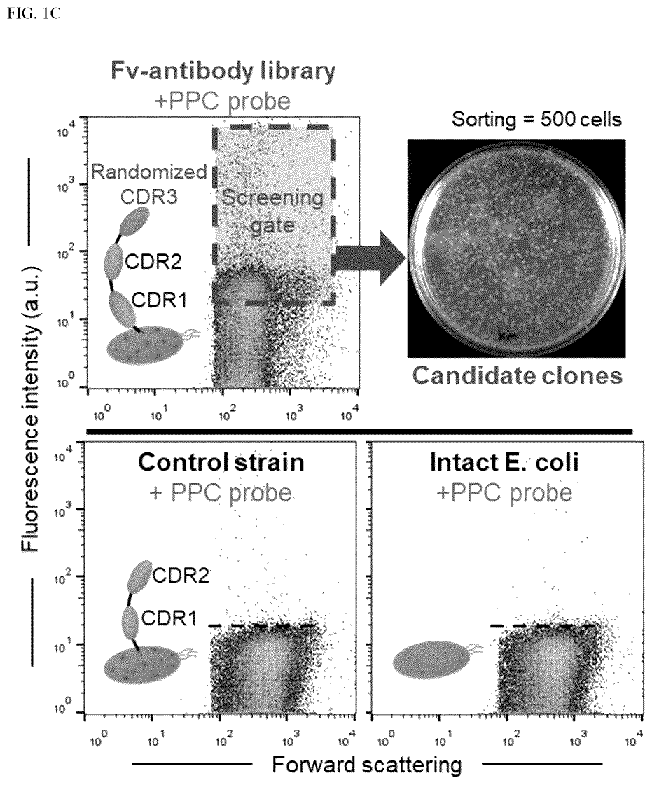

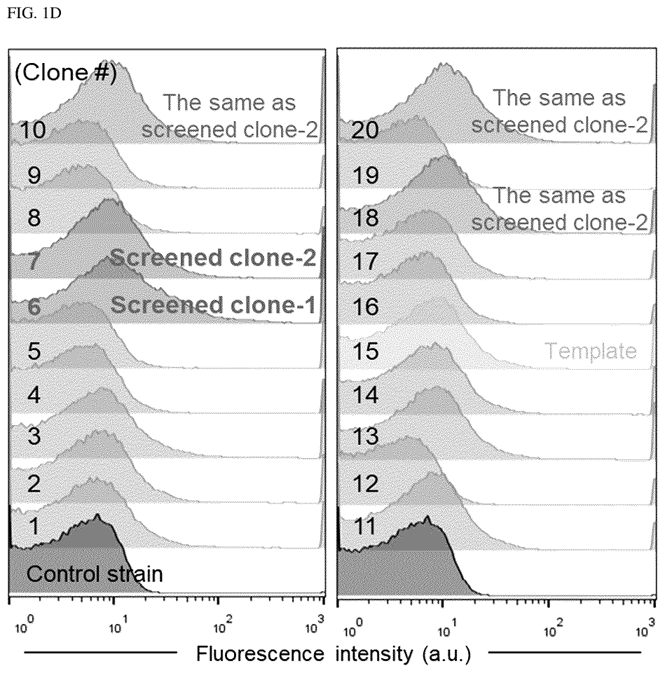

In the present disclosure, Fv-antibodies with a high binding affinity to PPC region were screened from Fv-antibody library to prevent the infection of SARS-CoV-2. As the first step, the PPC site was expressed as a screening probe of the Fv-antibody library. Among the amino acid sequence of SP (1,273 residue), the PPC region including S1/S2, and S2′ sites (amino acid sequence of 661-900, 240 residues) were expressed as a fusion protein with GFP (53.6 kDa) as shown in FIG. 1B. The screening of Fv-antibodies against PPC region was carried out by isolating the E. coli clones bound with the PPC probe using flow cytometry. As shown in FIG. 1C, the dot plots of Fv-antibody library showed a highly fluorescent region in comparison with control strain (only with autodisplayed CDR1 and CDR2) and intact E. coli, which represented the E. coli clones with the binding affinity to PPC region. The E. coli clones with a high fluorescence were isolated and cultured on an agar plate. Then, the clones with a high binding affinity to the PPC probe were selected and the final clones were determined after the oligonucleotide sequence of CDR3 region. As shown in FIG. 1D, two clones were determined to be the final clones with target Fv-antibodies against the PPC region. The detailed information of these Fv-antibodies such as oligonucleotide sequences, corresponding amino acid sequences and the binding affinity was summarized in Table 1.

Table 1 shows the oligonucleotide and amino acid sequences of the Fv-antibody and the binding affinity (KD) thereof to the PPC region.

TABLE 1

| | | | Binding | |
|---|---|---|---|---|
| | CDR3 region sequence | | constant | |
| | | Amino | (K_{D}) | |
| Screened | Oligonucleotide | acid | FACS | SPR |
| clone | (33 bp) | (11 mer) | (nM) | (nM) |
| 1 | 5′-GAC GGT CGT GCT | ¹DGRAR | 191.6 | 38.0 |
| | CGC CAA AGC CAG | ⁵QSQDD | | |
| | GAT GAT GAT-3′ | ¹¹D | | |
| 2 | 5′-GGC AGC CAA ATC | ¹GSQIA | 343.4 | 42.9 |
| | GCT CTT CGG CGG | ⁵LRRRD | | |
| | AGG GAT TTC-3′ | ¹¹F | | |

### Binding Analysis of Fv-Antibodies

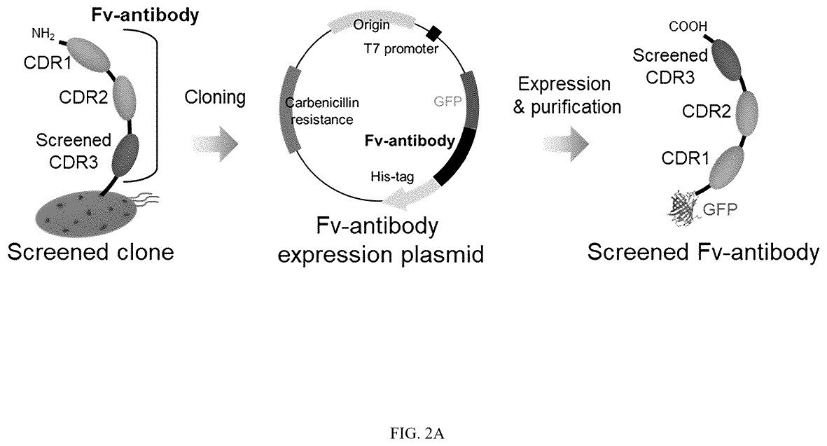

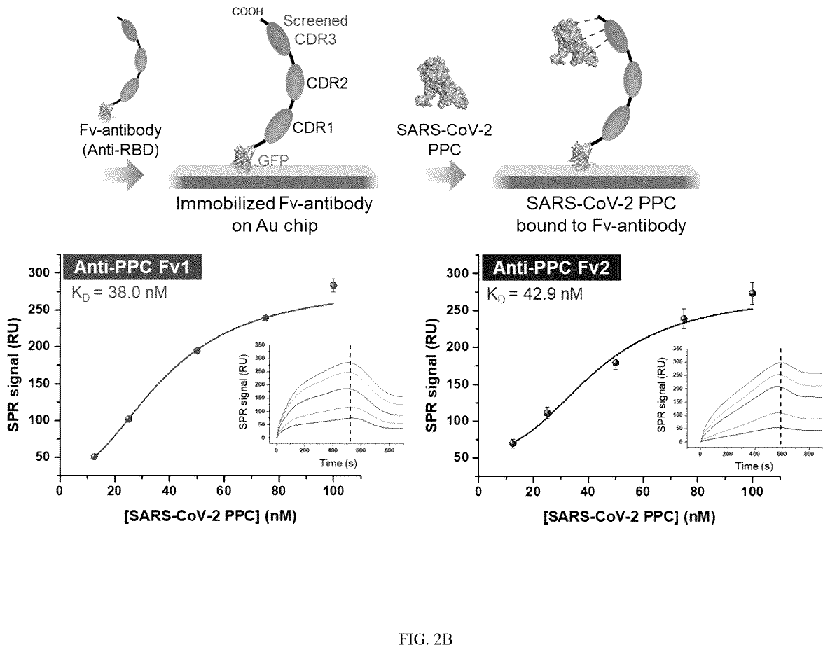

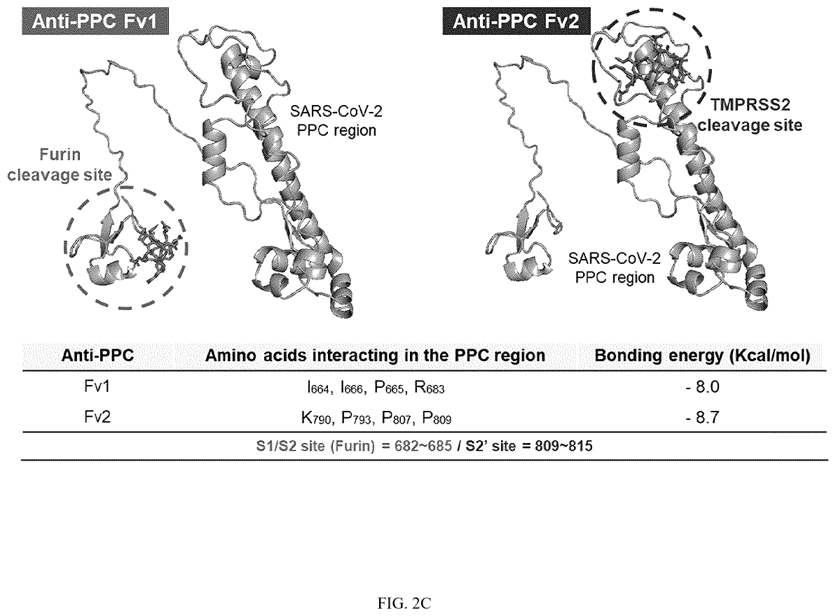

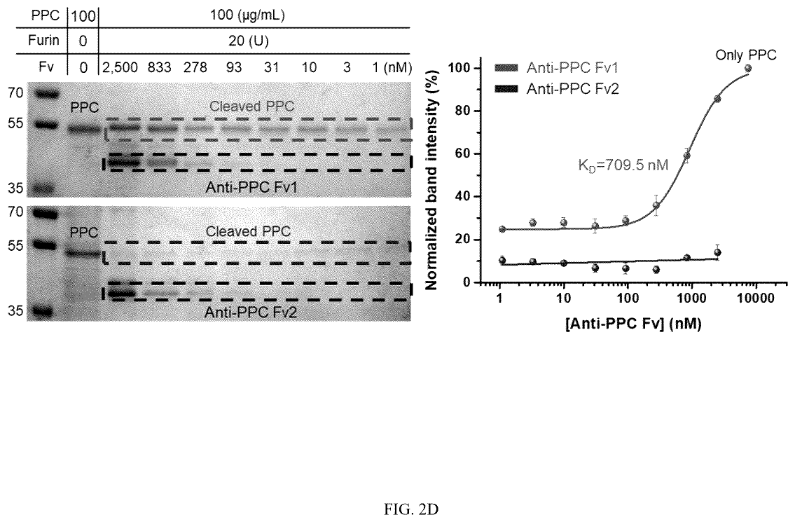

The screened Fv-antibodies were expressed as soluble proteins with GPF as shown in FIG. 2A. The binding affinity of expressed Fv-antibodies were estimated to be 38.0 nM for Fv-1 and 42.9 nM for Fv-2 using a SPR biosensor with immobilized PPC probe on Au chip as shown in FIG. 2B. As the binding affinity of the enzyme, furin for the cleavage of S1/S2 region was known to be 16 nM, 18 the binding affinity of Fv-antibodies were considered to be high enough to effectively bind to the targeted PPC region. In order to analyze the binding region of each Fv-antibody, docking simulation was carried out using Autodock vina from Scripps Research (La Jolla, CA, USA) and the structure of PPC region of SP (PDF 6ZP5). As shown in FIG. 2C, Fv-antibodies were analyzed to bind at different region of SP with the binding energy (ΔG) of −8.0 kcal/mol for Fv-1 and −8.7 kcal/mol for Fv-2 which represented the spontaneous binding of Fv-antibodies to the PPC region. Each Fv-antibody was analyzed to cleavage sites for different proteolytic enzymes. The binding region of Fv-1 was found to include the cleavage site for furin (residues: 682-685) and Fv-2 was analyzed to bind to the cleave site for TMPRSS2 (residues: 809-815). The binding of Fv-antibodies at the analyzed sites was investigated by performing the cleavage reaction with PPC probe and furin after the treatment of each Fv-antibody. The cleavage reaction was carried out by changing the concentration of Fv-antibodies at the constant concentration of PPC probe and furin. As shown in FIG. 2D, the PPC probe (the protein band at 53.6 kDa) was found to be not cleaved by furin which represented the blockage of S1/S2 region by the binding of Fv-1. However, when Fv-2 was treated, the PPC probe was found to be cleaved. The cleavage reaction of furin was active even if Fv-2 was treated. As previously mentioned, Fv-2 binding to the cleavage site of TMPRSS2 and the cleavage reaction of furin could not be blocked by treatment of Fv-2 to the PPC probe. These results showed that Fv-antibodies were bound at different cleavage sites at PPC region for different proteolytic enzymes.

### Estimation of Neutralizing Activity of Fv-Antibodies

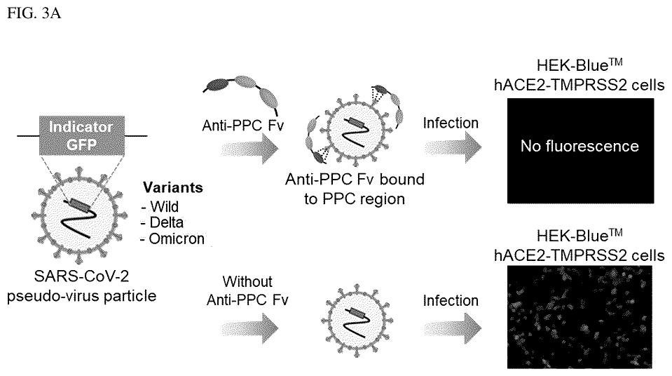

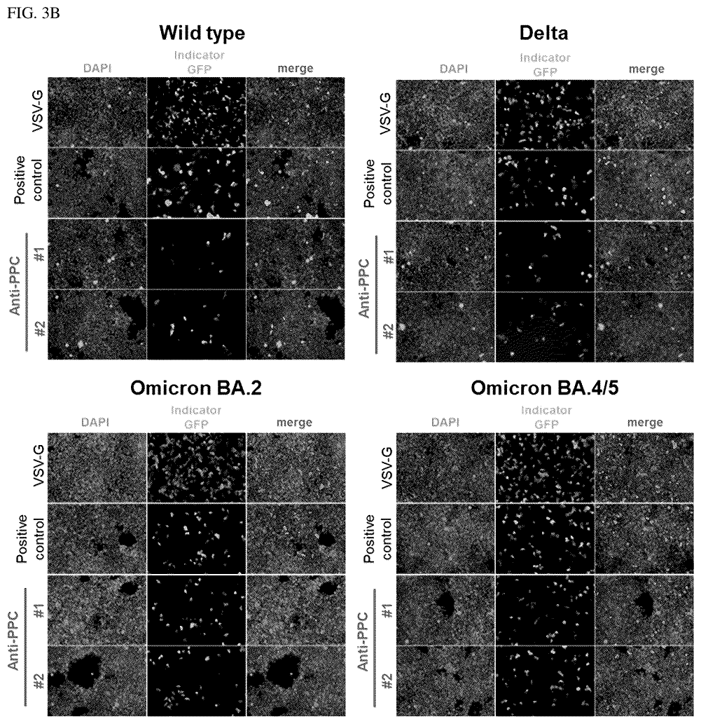

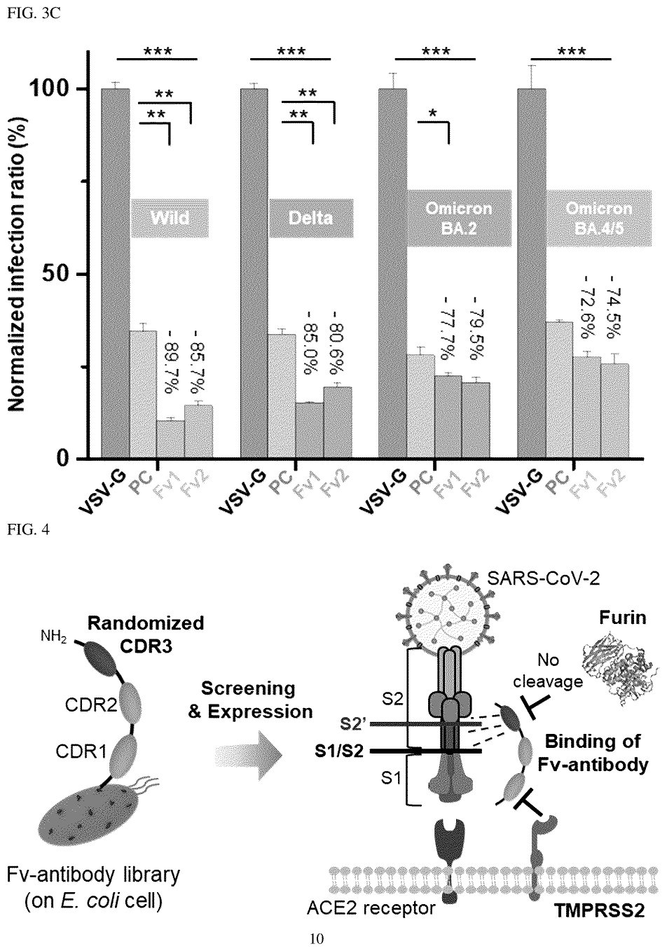

The neutralizing activity of Fv-antibodies was estimated using a cell-based infection assay using pseudo-virus particles. As a host cell for the infection assay, a HEK293 cell line with stably expressed human ACE2 receptor and TMPRSS2 was used. Further, pseudo-virus particle was prepared using lenti-virus with the expressed spike proteins of SARS-CoV-2 which corresponded to Wu-1 (wild type), Delta, Omicron (BA.2), and Omicron (BA.4/5). The pseudo-virus particles had the expression vector for green fluorescence protein (GFP) which was expressed when the pseudo-virus particle was infected into the host cell. As shown in FIG. 3A, the fluorescence signal could be detected from the host cells when the infection was made by the pseudo-virus particle. When Anti-PPC Fv could prevent the infection of virus, no fluorescence signal was detected. Therefore, the prevention activity of Fv-antibodies from the pseudo-virus particle infection, that is, the neutralizing activity of Fv-antibodies could be estimated from the fluorescence of host cells. As shown in FIG. 3B, the cell-based infection assay could be carried out using four kinds of pseudo-virus particles with the expressed spike proteins of SARS-CoV-2 which corresponded to Wu-1 (wild type), Delta, Omicron (BA.2), and Omicron (BA.4/5). When two kinds of Fv-antibodies were used in the cell-based infection assay, the fluorescence signal was apparently reduced from the host cells. These results indicated that these Fv-antibodies had the neutralizing activity of pseudo-virus particle infection. As shown in FIG. 3C, the neutralizing activity was statistically estimated by measurement of fluorescence signal from host cells using flow cytometry. The neutralizing activity against pseudo-virus particle with SP of Wu-1 type was estimated to be −89.7% for Fv-1 and −85.7% for Fv-2 in comparison with VSV-G as a positive control of infection (100%). The high neutralizing activity of Fv-1 was attributed to the fact that Fv-1 could bind to the cleavage site for furin (FIG. 2C). These results showed that Fv-1 could effectively prevented the activity of furin through the binding to the cleavage site for this proteolytic enzyme. The neutralizing activity of two Fv-antibodies were estimated to be similar level (−70% to −80%) for other kinds of pseudo-virus particle. These results showed that the neutralizing antibodies against the PPC site could be effectively used for the prevention of SARS-CoV-2 because corona virus family had a highly conserved amino acid sequence at the PPC site.

TABLE 2

| Sequence | | | | |
|---|---|---|---|---|
| ID | Sequence | Molecule | | |
| Number | Name | Type | Organism | Sequence |
| 1 | peptide_1 | AA | Synthetic | DGRARQ |
| | | | construct | SQDDD |
| 2 | peptide_2 | AA | Synthetic | GSQIAL |
| | | | construct | RRRDF |
| 3 | CDR1 | AA | synthetic | TYGIQ |
| | | | construct | |
| 4 | CDR2 | AA | synthetic | WIHAG |
| | | | construct | TGGTK |
| | | | | YSRKF |
| | | | | QG |

### Binding of Fv-Antibody to S1/S2 Cleavage Site of SARS-CoV-2 Spike Protein

FIG. 4 is a diagram showing the binding process of the Fv antibody to the specific site of the SARS-CoV-2 spike protein. FIG. 4 visualizes the binding of the Fv antibody to the S1/S2 cleavage site of the SARS-CoV-2 spike protein. Referring to FIG. 4, when the Fv antibody binds to the spike protein of the virus, the amino acid sequence of the CDR3 region effectively binds to the S1/S2 cleavage site of the spike protein, thereby forming a structure capable of interfering with additional protein cleavage by TMPRSS2. The principle that the CDR3 region of the Fv antibody contributes to binding of the Fv antibody to the specific site of the spike protein may be identified.

Although the embodiments of the present disclosure have been described above with reference to the accompanying drawings, the present disclosure may not be limited to the embodiments and may be implemented in various different forms. Those of ordinary skill in the technical field to which the present disclosure belongs will be able to appreciate that the present disclosure may be implemented in other specific forms without changing the technical idea or essential features of the present disclosure. Therefore, it should be understood that the embodiments as described above are not restrictive but illustrative in all respects.

What is claimed is:

1. A peptide capable of specifically binding to a PPC (proprotein convertase) region of a SARS-CoV-2 spike protein, wherein the peptide comprises a peptide sequence of SEQ ID NO:
1 or a peptide sequence of SEQ ID NO: 2:
[SEQ ID NO: 1]
DGRARQSQDDD
[SEQ ID NO: 2]
GSQIALRRRDF.

2. The peptide of claim 1, wherein the peptide binds to the PPC region of the SARS-CoV-2 spike protein to prevent cleavage of the spike protein by TMPRSS2 of an infection target host cell.

3. A nucleic acid coding the peptide according to claim 1.

4. A recombinant expression vector including the nucleic acid according to claim 3.

5. A cell transformed with the recombinant expression vector according to claim 4.

6. The cell of claim 5, wherein the cell includes at least one cell selected from the group consisting of animal cells, plant cells, yeast, E. coli, and insect cells.

7. The cell of claim 6, wherein the cell includes at least one cell selected from the group consisting of COS7 (monkey kidney cells) cells, NSO cells, SP2/0 cells, CHO (Chinese hamster ovary) cells, W138, BHK (baby hamster kidney) cells, MDCK, myeloma cell lines, HuT 78 cells and HEK293 cells, Escherichia coli, Bacillus subtilis, Streptomyces sp., Pseudomonas sp., Proteus mirabilis or Staphylococcus sp., Aspergillus sp., Pichiapastoris, Saccharomyces cerevisiae, Schizosaccharomyces sp., and Neurosporacrasa.

8. An antibody or antigen-binding fragment thereof capable of specifically binding to a PPC region of a SARS-CoV-2 spike protein, wherein the antibody includes a Fv antibody, wherein a CDR3 region of the Fv antibody includes a peptide sequence of SEQ ID NO: 1 or a peptide sequence of SEQ ID NO: 2:
[SEQ ID NO: 1]
DGRARQSQDDD
[SEQ ID NO: 2]
GSQIALRRRDF.

9. The antibody or antigen-binding fragment thereof of claim 8, wherein the antibody or antigen-binding fragment thereof binds to the PPC region of the SARS-CoV-2 spike protein, thereby interfering with cleavage of the spike protein by TMPRSS2 of an infection target host cell.

10. A composition for preventing SARS-CoV-2 infection, the composition comprising the antibody or antigen-binding fragment thereof according to claim 8.
